(19) United States
(12) Patent Application Publication
(10) Pub. No.: US 2016/0096027 A1
(43) Pub. Date: Apr. 7, 2016

(54) WIRELESS DELIVERY OF TRANSCUTANEAOUS ELECTRICAL NERVE STIMULATION (TENS) TREATMENTS

(71) Applicants: Yaakov ASSEO, Dallas, TX (US); Liran GRENWALK, Lexington, KY (US)

(72) Inventors: Yaakov ASSEO, Dallas, TX (US); Liran GRENWALK, Lexington, KY (US)

(21) Appl. No.: 14/503,413

(22) Filed: Oct. 1, 2014

(51) Int. Cl. A61N 1/372; A61N 1/378; G08C 17/02; A61N 1/36

(52) U.S. Cl. CPC A61N 1/37223; A61N 1/36014; A61N 1/378; G08C 17/02

(57) ABSTRACT

A method and system for performing transcutaneaous electrical nerve stimulation (TENS) via a portable mobile device. The TENS system includes a wireless radio, a microcontroller, and a switch. The wireless radio is compliant with a wireless networking standard, and is configured to wirelessly receive, in accordance with the wireless networking standard, instructions derived from a first treatment plan selected from one or more treatment plans, wherein each treatment plan of the one or more treatment plans respectively specifies characteristics of an electrical current including a desired pulse and timing associated with the treatment plan. The microcontroller is configured to process the instructions derived from the first treatment plan, and output settings specific to the processed instructions. The switch is configured to, based on the settings specific to the processed instructions, deliver the electric current to the patient at the desired pulse and timing as specified by the first treatment plan.

### TECHNICAL FIELD

The present disclosure relates generally to transcutaneaous electrical nerve stimulation (TENS) devices, and more particularly to techniques for performing transcutaneaous electrical nerve stimulation via a portable mobile device.

### BACKGROUND

Transcutaneaous electrical nerve stimulation (TENS) is a non-invasive, method of relieving pain without the administration of any drug medication, and is primarily used for the symptomatic or chronic non-malignant management of chronic pain. During transcutaneaous electrical nerve stimulation, a TENS unit (or module) generates pulsed currents which are applied to the surface of a patient's skin via conducting pads called electrodes, and the stimulating pulses help prevent pain signals from reaching the patient's brain. Providing adequate dosing (e.g., amplitude, pulse width and frequency) along with moderated intensity is critical in obtaining the maximum pain relief possible.

Many TENS devices are stationary and are located at a physician's office which can become inconvenient for patients. Treatment for ailments such as back pain can require an hour or so of daily treatment for a period of several months. There are some mobile TENS devices that are typically battery powered and allow for treatment at home or elsewhere away from the physician's office. However, these conventional devices are often limited to just a few settings which are selectable by a manual switch or other simplistic interface.

What is needed is a TENS device that leverages the power of wireless computing devices and mobile applications while retaining the convenience of mobility. Furthermore, the device should leverage resources available on the Internet through the computing devices.

### BRIEF SUMMARY

In general, this specification describes a method, computer software, and a system for performing transcutaneous electrical nerve stimulation (TENS) via a portable mobile device. In one implementation, the transcutaneaous electrical nerve stimulation (TENS) system includes an enclosure that includes a wireless radio, a microcontroller, and a switch. The wireless radio is compliant with a wireless networking standard (e.g., Bluetooth, Wi-Fi, IEEE 802.15.4, etc.), and is configured to wirelessly receive, in accordance with the wireless networking standard, instructions derived from a first treatment plan selected from one or more treatment plans. Each given treatment plan of the one or more treatment plans respectively specifies characteristics of an electrical current including a desired pulse and timing associated with the given treatment plan. The microcontroller is configured to (i) process the instructions derived from the first treatment plan, and (ii) output settings specific to the processed instructions. The switch is configured to, based on the settings specific to the processed instructions, deliver the electric current to the patient at the desired pulse and timing as specified by the first treatment plan.

Implementations may provide one or more of the following advantages. Unlike a conventional TENS module that can only apply a limited (or fixed) number of pre-defined settings (e.g., for pulse width, pulse length, intensity, and so on) while performing transcutaneaous electrical nerve stimulation, a TENS module of the present disclosure is configured to (i) receive one or more treatment plans, each specifying an electrical profile, via a wireless communication device, and (ii) drive one or more electrodes in accordance with the electrical profile specified by a given treatment plan. Accordingly, an potentially unlimited number of treatment plans can be received by a TENS module of the present disclosure to provide a highly customized treatment plan to a target patient.

The details of one or more implementations are set forth in the accompanying drawings and the description below. Other features and advantages will be apparent from the description and drawings, and from the claims.

### BRIEF DESCRIPTION OF THE DRAWINGS

FIG. 1A is a block diagram of a transcutaneaous electrical nerve stimulation (TENS) system including a TENS module wireless coupled to a wireless communication device, in accordance with an implementation of the present disclosure.

FIG. 1B is a block diagram of a TENS system including a TENS module powered by a mobile phone and receiving wireless data communications from the mobile phone, in accordance with another implementation of the present disclosure.

FIG. 1C is a block diagram of a TENS system including wireless electrodes, in accordance with an implementation of the present disclosure.

FIG. 2 illustrates a method for performing TENS treatments using wireless communications, in accordance with an implementation of the present disclosure.

FIG. 3 is a more detailed block diagram of internal electrical components of a TENS module, in accordance with an implementation of the present disclosure.

FIG. 4 is a schematic diagram illustrating a user interface of a mobile application for driving a TENS module, in accordance with an implementation of the present disclosure.

FIG. 5 is a block diagram illustrating a network architecture for delivering one or more treatment plans to a user, in accordance with an implementation of the present disclosure.

FIG. 6 is a schematic diagram illustrating a TENS system including a TENS module utilized as part of a stand for a mobile device, in accordance with an implementation of the present disclosure.

Like reference symbols in the various drawings indicate like elements.

### DETAILED DESCRIPTION

Methods, computer software, and systems for performing transcutaneous electrical nerve stimulation (TENS) via a portable mobile device, are disclosed herein. A TENS module can be a self-sufficient device, can be driven by a host wireless device such as a mobile telephone, or can be driven by a remote server. The description is non-limiting and embodiments are provided as examples of broader principles that can be applied to many other embodiments not explicitly set forth.

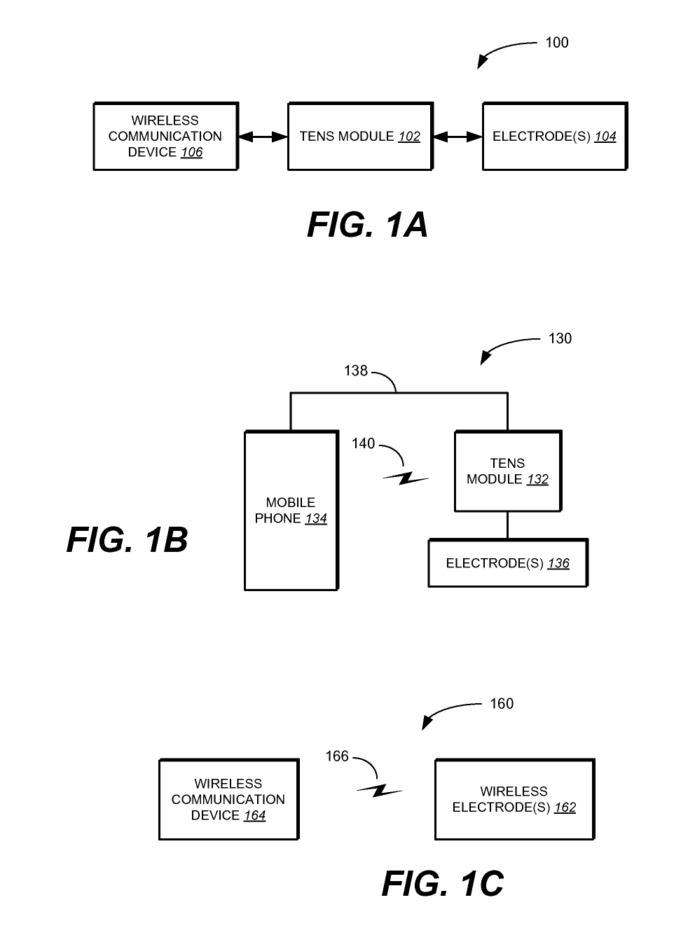

FIG. 1A illustrates one implementation of a TENS system 100 including a TENS module 102 in communication with (i) a wireless communication device 106 and (ii) one or more electrodes 104. Unlike a conventional TENS module that can only apply a limited (or fixed) number of pre-defined settings (e.g., for pulse width, pulse length, intensity, and so on) while performing treatments, the TENS module 102 is configured to (i) receive one or more treatment plans, each specifying an electrical profile as discussed in greater detail below, via the wireless communication device 106, and (ii) drive the one or more electrodes 104 in accordance with the electrical profile specified by a given treatment plan.

The one or more treatment plans received by the TENS module 102 can be stored in a memory (not shown) of the TENS module 102 and/or a memory (not shown) of the wireless communication device 106. The electrical profile specified by a given treatment plan can include one or more settings or characteristics associated with generating pulsed currents to be applied by the electrode(s) 104 to the skin of a patient. In one implementation, the settings include one or more of speed, intensity, duration, pulse width, pulse length, pattern, frequency, and so on). Accordingly, the TENS module 102 can potentially drive the one or more electrodes 104 in accordance with an unlimited number of treatment plans, including treatment plans (having electrical profiles) customized to a particular patient (or user) to obtain the maximum pain relief possible for the patient. The one or more electrodes 104 include pads (not shown) that can be placed on the skin of a patient to deliver electrical current at a desired location. In one implementation, the TENS module 102 can accommodate a number of electrodes/pads in pairs or individually separated.

In the implementation of FIG. 1A, the TENS module 102 is a standalone unit that is separate from the wireless communication device 106 (see e.g., FIG. 1B. In such an implementation, the TENS module 102 is powered by an electrical outlet and/or batteries. In another implementation, the TENS module 102 is attachable to the wireless communication device 106 via a communication port of the wireless communication device 106 (see e.g., FIG. 1B). For example, the communication port can be, for example, an audio port or a Universal Serial Bus (USB) port. In such an implementation, the TENS module 102 can be powered via the wireless communication device 106 through the communication port.

The wireless communication device 106 executes a treatment file that generates specific settings wirelessly transmitted to the TENS module 102. For example, a mobile application, an on-demand application, a web-based application, or widget comprises source code related to specific treatments and peripheral information. In one implementation, the wireless communication device 106 is a cloud-based server that transmits to the TENS module 102 capable of an Internet, 3G, 4G, or other direct or indirect connection to a data network backbone without an intervening mobile application.

The wireless communication device 106 can be any device with one or more radio frequency (RF) antennae capable of performing wireless communication in accordance with a wireless communication standard. For example, the wireless communication device can be a desktop computer, a laptop computer, a tablet, a phablet, a smart phone, an access point, remote server, or other processor-driven device capable of wirelessly communicating with another device. The wireless communication device 106 can further comprise a processor, a memory, an operating system, a mobile application, a display, an audio jack, and other standard computing components. Various wireless communication standards include the IEEE 802.11 wireless networking standards (Wi-Fi), Bluetooth, Bluetooth Low Energy (BLE), the IEEE 802.15.4 wireless networking standard (e.g., ZigBee or 6LoWPAN), the IEEE 802.16 wireless networking standards (WiMAX), the near-field communication (NFC) standard, and the like.

FIG. 1B illustrates a TENS system 130 including a TENS module 102 in communication with (i) a mobile phone 134 and (ii) one or more electrodes 136. In the implementation shown in FIG. 1B, the TENS module 132 is coupled to the mobile phone 134 via a (physical) connection 138 to receive power from the mobile phone 134 via a communication port of the mobile phone 134. The power drawn from the mobile phone 134 is used, in one implementation, to drive the one or more electrodes 136. In one implementation, the communication port of the mobile phone 134 comprises an audio port, and the connection 138 comprises a standard 3.5 mm three or four conductor audio input jack that can be inserted into the audio port (not shown) of the mobile phone 134. In another implementation, the communication port of the mobile phone 134 comprises a USB port, and the connection 138 comprises a USB plug (e.g., a micro USB plug) that can be inserted into the USB port of the mobile phone 134. One or more battery packs can be used to supplement power to the TENS module 132 and/or the electrode(s) 136. In one implementation, the TENS module 132 receives streaming video and audio associated with a given treatment plan from the mobile phone 134 via wireless communication 140. Additionally, the electrical profile associated with a given treatment plan is also transmitted from the mobile phone 134 to the TENS module 132 via wireless communication 140. The wireless communication 140 can be compliant with a wireless networking standard as mentioned above.

FIG. 1C illustrates a TENS system 160 including wireless electrodes 162. In one implementation, one or more components of a TENS module (e.g., TENS module 102) is integrated with each wireless electrode 162. In such an embodiment, the TENS system 160 includes one or more untethered and decentralized TENS pads having a slim profile battery and wireless radio integrated with the wireless electrodes 162 to permit the wireless electrodes 162 to wirelessly receive instructions from a wireless communication device 164 while being untethered (e.g., not physically connected) to the wireless communication device 164. In one implementation, the wireless communication device 164 is a mobile phone, or other wireless communication device as discussed above. In another implementation, the wireless communication device 164 is a TENS module (or unit) having a wireless radio configured to communicate with the wireless radio integrated within each of the wireless electrodes 162. In the TENS module implementation, a further wireless connection as described in FIGS. 1A and 1B is possible. A mobile application on the wireless communication device 164 in this implementation acts as a server for several clients (e.g., wireless electrodes 162 or one or more TENS modules 102). Wi-Fi can be implemented to easily handle the multiple RF connections, or point-to-point Bluetooth connections can be managed in order to provide necessary information to each of the clients, or wireless electrodes in a round-robin or as needed basis through push or pull data transfers.

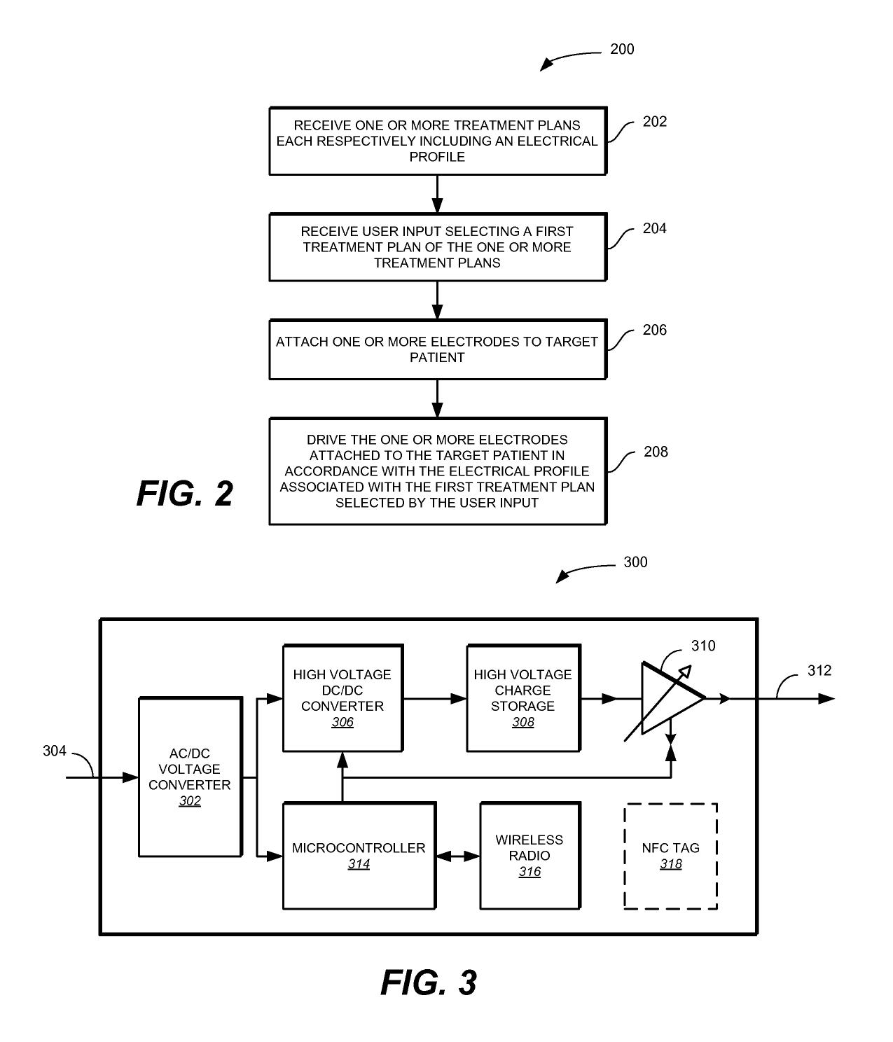

FIG. 2 illustrates a method 200 for performing transcutaneaous electrical nerve stimulation in accordance with an implementation of the present disclosure. One or more treatment plans, each respectively including an electrical profile, is received by a TENS module (e.g., TENS module 102) (step 202). The treatment plans can be executed by a mobile application on a wireless communication device and communicated to a TENS module, or even executed on a remote server that streams directly to a TENS module. As discussed above, the electrical profile specified by a given treatment plan (e.g., in a treatment file) can include one or more settings or characteristics associated with generating pulsed currents to be applied by one or more electrodes (e.g., electrode(s) 104) to the skin of a patient. In one implementation, the settings include one or more of speed, intensity, duration, pulse width, pulse length, pattern, frequency, and so on). A treatment plan dictated by a treatment file can be infinitely variable. Treatments can span just one session or multiple sessions. Follow-up sessions may vary as treatments intensify or wind down. Patients can use a touch screen for manipulation of settings during treatments or for manipulating schedules of sessions, or the like. In one embodiment, a third-party such as a physician updates treatments that are automatically downloaded to patients.

A treatment plan can further include multimedia (e.g., an audio file and/or a video file) that can be played or viewed on a display associated with a wireless communication device (e.g., wireless communication device 106) or the TENS module. The multimedia associated with a given treatment plan can include specific instructions for properly positioning one or electrodes on the body of a patient to achieve a desired effect associated with the treatment plan. The file can further include information associated with a specific use case for the given treatment plan. For example, a specific use case for a particular treatment plan may be to provide remedy for lower back pain, and an audio/video file associated with the treatment plan can include instructions for locating electrodes on the lower part of the back of the patient to achieve the best results for remedying the lower back pain. Multimedia can be simply mood music selected for relaxation. Additionally, Internet links can be provided for further treatment resources.

User input is received selecting a first treatment plan of the one or more treatment plans (step 204). In one implementation, the user input is received via user input applied to the wireless communication device. For example, in one implementation in which the wireless communication device comprises a mobile phone, the user input can be received via input on a touch-screen of the mobile phone. In another implementation, the user input is received via user input applied to the TENS module—e.g., in an implementation in which the wireless communication device is an access point, and the TENS module is a standalone unit that is separate from the access point. In one implementation, one or more settings of the electrical profile associated with the first treatment plan can be modified by the user based on user input received via the wireless communication device and/or the TENS module.

In another embodiment, third-parties can make real-time manipulations to treatments in session based on feedback from patients. For example, a bio-sensor attached to a TENS module can provide blood pressure, pulse information or other bio-information that is sent to a remote party for display. In response, settings can be adjusted.

One or more electrodes (e.g., electrode(s) 104) are attached to a target patient (step 206). In general, each of the one or more electrodes comprises one or more (disposable or re-usable) pads that can be applied to the skin of the target patient. In one implementation, the one or more electrodes are attached onto a target patient at one or more locations as specified or indicated by a treatment file associated with the first treatment plan.

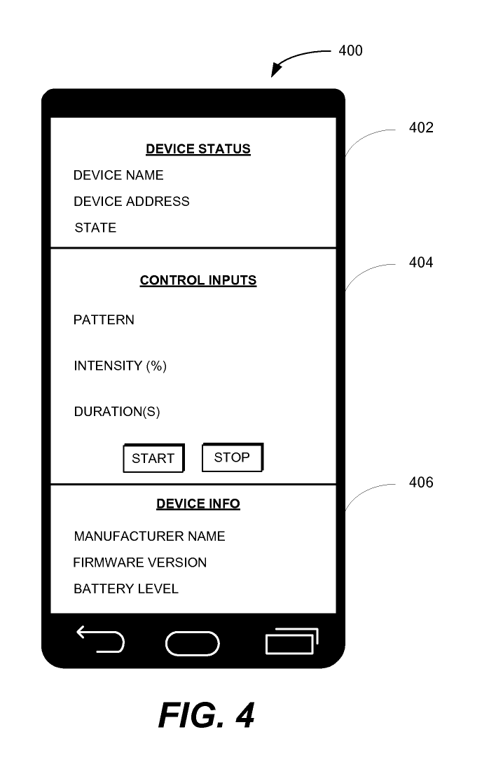

The one or more electrodes attached to the target patient are driven, e.g. by the TENS module, in accordance with the electrical profile associated with the first treatment plan selected by the user input (step 208). One or more settings associated with the electrical profile can be altered based on user input via a user interface as described in connection with FIG. 4 below.

FIG. 3 is a block diagram of a transcutaneaous electrical nerve stimulation (TENS) module 300 in accordance with an implementation of the present disclosure. The TENS module 300 includes (within an enclosure) an alternating current/ direct current (AC/DC) voltage converter 302 (power supply), a high voltage DC/DC converter 306, a high voltage charge storage 308, an amplifier/switch 310, a microcontroller 314, a wireless radio 316, and a near field communication (NFC) tag 318. In one implementation, the AC/DC voltage converter 302 generates a DC source from a software generated audio tone input 304 (received via an audio jack (not shown) of the TENS module 300). Based on the DC source received from the AC/DC voltage converter 302, the high voltage DC/DC converter 306 provides a variable amplitude high voltage, low current source to the high voltage charge storage 308. The high voltage charge storage 308 accumulates charge received from the high voltage DC/DC converter 306. In one implementation, the high voltage charge storage 308 comprises a storage element such as a capacitor or super capacitor. The amplifier/switch 310 generates an output 312 for driving one or more electrodes in accordance with an electrical profile specified by a given treatment plan. The microcontroller 314 monitors the modes of operation of the TENS module 300, and controls the amplifier/switch 310 such that the output 312 of the amplifier/switch 310 has a desired frequency, pattern, and charge as specified by a given treatment plan. In one implementation, the microcontroller 314 processes instructions received by the wireless radio 316, and outputs specific settings (based on the processed instructions) to control the amplifier/switch 310. The wireless radio 316 provides for wireless communication between the TENS module 300 and a wireless communication device (e.g., mobile phone 134 of FIG. 1B). In one implementation, the wireless radio 316 comprises a Bluetooth low energy radio. In one implementation, each of the microcontroller 314 and the wireless radio 316 are powered by the DC source generated by the AC/DC voltage converter 302. In one implementation, each microcontroller 314 and the wireless radio 316 are both embedded on a common substrate (e.g., on a system on a chip integrated circuit or SoC IC).

The TENS module 300 optionally includes an NFC tag 318 that can direct a wireless communication device (e.g., mobile phone 134) to a URL at which a mobile application can be downloaded to control various settings of the TENS module 300 via the microcontroller 314 and, therefore, to control various characteristics of the output 312 (e.g., frequency, pattern, charge, and so on). In lieu of an NFC tag, the TENS module 300 can include a quick response (QR) code (e.g., printed on an outside surface of a casing of the TENS module 300) that directs a user to a URL at which a mobile application can be downloaded.

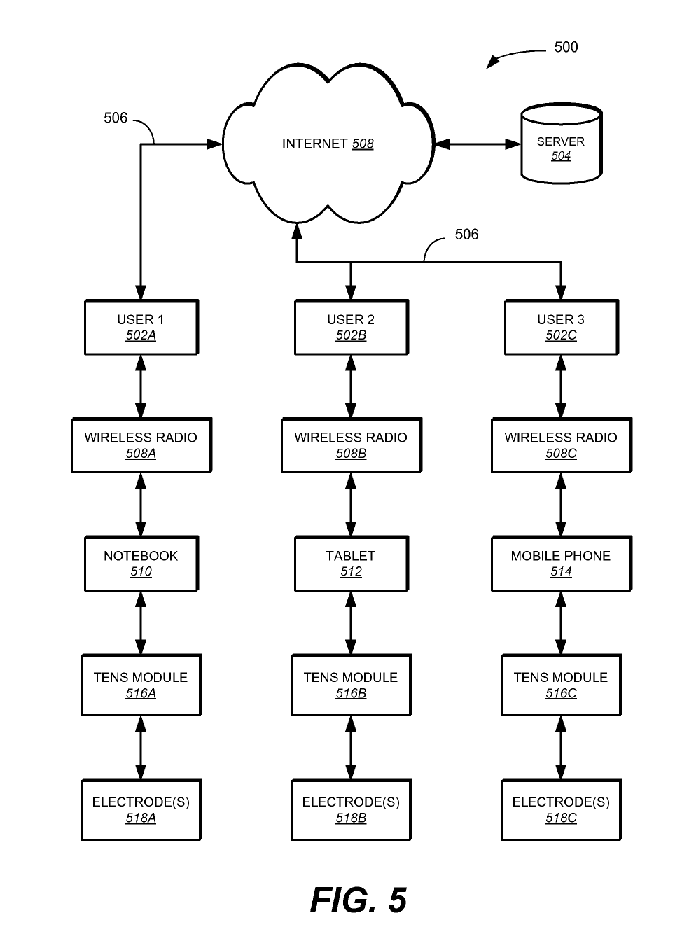

FIG. 4 illustrates an implementation of a user interface 400 of a mobile application for driving a TENS module (e.g., TENS module 300 of FIG. 3). In FIG. 4, the user interface 400 is shown displayed on a display of a mobile phone (e.g., mobile phone 134 of FIG. 1B). In one implementation, the mobile application is responsible for driving the TENS module. For example, in such an implementation, the mobile application controls the amplitude, mode, frequency, and timing of the dosage of electrical current delivered to a target patient. The mobile application can further permit a TENS module to be driven by a customized electrical profile, and provide step-by-step instructions of how to properly use a TENS module to achieve the maximum benefit of a desired treatment. In one implementation, the mobile application communicates with a cloud server application (as discussed in connection with FIG. 5 below), and can suggest new treatment plans based on the history of a patient's utilization of a TENS module and associated mode of operation. Cloud services can also push down new offers, treatment plans, and so on, based on a patient's specific history of utilization of a TENS module and associated mode of operation.

As shown in FIG. 4, the user interface 400 includes a first section 402 corresponding to device status, a second section 404 corresponding to control inputs, and a third section 406 corresponding to device information. The first section 402 includes status associated with the TENS module associated with the mobile phone. Example status of a TENS module can include—a name (device name) of the TENS module, an address (device address) of the TENS module, and a state of the TENS module (e.g., whether a TENS module is “connected” or “disconnected” with the mobile phone). The second section 404 includes inputs (control inputs) for controlling a TENS module. Example inputs include “Pattern” (e.g., circular, or the like), “Intensity (%)”, and “Duration”. In one implementation, a corresponding pop-up dialog box (not shown) will be displayed based on a user respectively clicking on the word “Pattern”, “Intensity”, or “Duration”, and the pop-up dialog box will respectively explain the various device patterns, intensities, durations and their intended uses. The third section 406 includes information corresponding to a manufacturer of an associated TENS module, a version of firmware being run by the associated TENS module, and a battery level associated with the mobile phone and/or the associated TENS module.

The user interface 400 can display other information associated with treatments, either directly from a treatment file or for other resources. As discussed above, multimedia can illustrate instructions for treatment, music videos can be played for relation, and web sites can be automatically accessed to provide certain information. In one implementation, a search engine is provided to allow a user to query for treatment files from Internet resources. A patient can select among search results for execution.

FIG. 5 illustrates an implementation of a network architecture 500 for delivering one or more treatment plans to users 502A-C (e.g., User 1, User 2, and User 3). In one implementation, the network architecture 500 includes a cloud server 504 from which a mobile application and treatment files can be downloaded via an Internet connection 506 from the Internet 508. The cloud server 504 further includes one or more treatments plans that can be respectively streamed (via wireless radios 508A-C) to wireless devices of the users 502A-C. In the example shown in FIG. 5, a mobile application and one or more treatment plans are delivered to a notebook 510 of User 1, a mobile application and one or more treatment plans are delivered to a tablet 512 of User 2, and a mobile application and one or more treatment plans are delivered to a mobile phone 514 of User 3.

In operation, the mobile application authenticates each user uniquely to services associated with the cloud server 504. Once authenticated, (in one implementation) the mobile application pushes or uploads, to the cloud server 504, a respective history of usage of the TENS modules 516A-C and electrodes 518A-C of users 502A-C. In one implementation, the history of usage includes use metrics including, but not limited to, times, durations, mode, amplitude, frequency, customizations of electrical profiles, and so on. The history of usage can be sent anonymously to the cloud server 504. Based on information received from the users 502A-C, the cloud server 504 can propose new treatment plans having modified electrical profiles, as well as propose location-related services and products based a respective location of each of the users 502A-C.

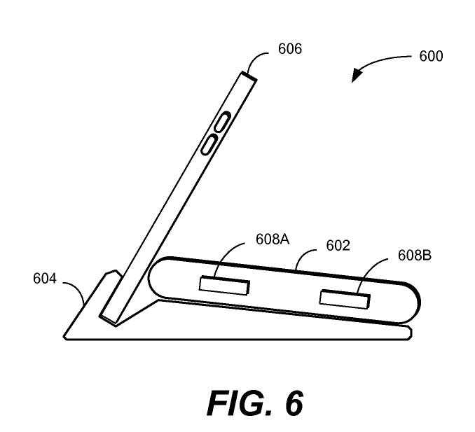

FIG. 6 illustrates an implementation of a TENS system 600 including a TENS module 602 utilized as part of a stand 604 for a mobile device 606. By utilizing the TENS module 602 as part of a stand for the mobile device 606, a user will be able to better manage the attachment of electrodes to ports 608A-B by having both the TENS module 602 and the mobile device 606 remain in a fixed position, which can prevent cords associated with the electrodes from being tangled.

One or more of method steps described above can be performed by one or more programmable processors executing a computer program to perform functions by operating on input data and generating output. Generally, various aspects of the present disclosure can take the form of an entirely hardware embodiment, an entirely software embodiment or an embodiment containing both hardware and software elements. In one implementation, various aspects of the present disclosure can be implemented in software, which includes but is not limited to firmware, resident software, microcode, etc. Furthermore, various aspects of the present disclosure can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD.

Various implementations for wirelessly delivering a controlled treatment of electrical current to a patient have been described. Nevertheless, various modifications may be made to the implementations. For example, though the techniques described above refer to transcutaneaous electrical nerve stimulation (TENS), the techniques are generally applicable to other forms of electrical stimulation—including electrical muscle stimulation, and the like. In addition, steps of the methods described above can be performed in a different order and still achieve desirable results. Accordingly, many modifications may be made without departing from the scope of the following claims.

What is claimed is:

1. A transcutaneous electrical nerve stimulation (TENS) system for wirelessly delivering a controlled treatment of electrical current to a patient, the TENS system comprising:
a wireless TENS unit, comprising:
an enclosure;
a wireless radio, compliant with a wireless networking standard, within the enclosure, wherein the wireless radio is configured to wirelessly receive, in accordance with the wireless networking standard, instructions derived from a first treatment plan selected from one or more treatment plans, wherein each given treatment plan of the one or more treatment plans respectively specifies characteristics of an electrical current including a desired pulse and timing associated with the given treatment plan;
a microcontroller within the enclosure, wherein the microcontroller is configured to (i) process the instructions derived from the first treatment plan, and (ii) output settings specific to the processed instructions; and
a switch within the enclosure, wherein the switch is configured to, based on the settings specific to the processed instructions, deliver the electric current to the patient at the desired pulse and timing as specified by the first treatment plan.

2. The TENS system of claim 1, wherein the wireless radio is compliant with one of : a Bluetooth protocol, a low energy Bluetooth protocol, an IEEE 802.11 protocol, a low energy IEEE 802.11 protocol, a low energy IEEE 802.15.4 protocol, and a cellular data protocol.

3. The TENS system of claim 1, further comprising:
a remote computing device in wireless communication with the wireless TENS unit, the remote computing device executing an application in accordance with a treatment file in order to generate the instructions transmitted to the TENS unit.

4. The TENS system of claim 3, wherein the remote computing device comprises at least one of: a smartphone, a laptop computer, a tablet computer, a wearable computing device, and a stationary computing device.

5. The TENS system of claim 3, wherein the remote computing device further comprises:
a display to show information associated with the one or more treatment plans.

6. The TENS system of claim 5, wherein the display shows a graphical representation of characteristics specified by the one or more treatment plans.

7. The TENS system of claim 3, wherein the remote communication device in communication with an external communication network, wherein the one or more treatment plans are stored in a server that is accessible by the remote communication device through the external communication network.

8. The TENS system of claim 1, wherein the remote communication device is a server device executing a web-based application and wirelessly communicates with the TENS unit through a network infrastructure.

9. The TENS system of claim 1, wherein the network infrastructure comprises at least one of: the Internet, a local access network, and a cellular data network.

10. The TENS system of claim 1, wherein the remote communication device in communication with a third party via an external communication network to receive from the third party instructions to modify the one or more treatment plans.

11. The TENS system of claim 1, further comprising:
electrodes, connected to the wireless TENS unit and attachable to a patient, to deliver the electric current output by the switch to the patient in accordance with the one or more treatment plans.

12. The TENS system of claim 11, wherein the electrodes further comprise a power supply and an RF radio, wherein the RF radio wirelessly connects to the TENS unit to receive instructions for generating the electrical output in accordance with the one or more treatment plans.

13. The TENS system of claim 1, wherein the wireless TENS unit further comprises:
a power supply to supply power to at least the microcontroller, the wireless radio, and the switch.

14. The TENS system of claim 1, wherein the wireless TENS unit further comprises:
a power supply input to receive power from an audio jack of an external device.
